(19) United States
(12) Patent Application Publication
(10) Pub. No.: US 2024/0173956 A1
(43) Pub. Date: May 30, 2024

(54) Method and Device for Manufacturing an Adherent Film Composite

(71) Applicant: LTS Lohmann Therapie-Systeme AG, Andernach (DE)

(72) Inventors: Peter Steinborn, Neuwied (DE); Sandra Wiedersberg, Steigra (DE); Dorothea Gutekunst, Montabaur (DE); Viktoria Steinbrecher, Andernach (DE)

(21) Appl. No.: 18/283,560

(22) PCT Filed: Mar. 17, 2022
(86) PCT No.: PCT/EP2022/057074

§ 371 Date: Sep. 22, 2023

(30) Foreign Application Priority Data
Mar. 23, 2021 (DE) 10 2021 107 208.5

(51) Int. Cl. B32B 43/00; B32B 38/18

(52) U.S. Cl. CPC B32B 43/006; B32B 38/1833; B32B 2255/26

(57) ABSTRACT

A method for manufacturing an adherent film composite, including the following steps: (i) providing a laminate, the laminate including a first film, a second film, and an adherent formulation arranged between the first film and the second film; (ii) feeding the laminate in a feeding direction; (iii) and separating the first film with the formulation arranged thereon from the second film. The films are separated by the first film being fed out obliquely to the feeding direction from the second film, wherein the second film is passed on in the feeding direction. The first film rotates about its longitudinal axis when feeding out the first film. Furthermore, a device for manufacturing an adherent film composite.

### CROSS REFERENCE TO RELATED APPLICATIONS

This application is the national phase of International Patent Application No. PCT/EP2022/057074 filed Mar. 17, 2022 and claims priority to German Patent Application No. 10 2021 107 208.5 filed Mar. 23, 2021, the disclosures of which are hereby incorporated by reference in their entireties.

### BACKGROUND OF THE INVENTION

### Field of the Invention

The present invention relates to a method and a device for manufacturing an adherent film composite, preferably a TTS film composite.

### Description of Related Art

For manufacturing film products, such as transdermal therapeutic systems (TTS) in particular, laminates are often used, preferably as intermediate products. In particular, such a laminate comprises a first film, e.g. a carrier film, a second film, e.g. a cover film, and a formulation arranged between the first and second films. The formulation preferably is a matrix. The formulation generally comprises an adhesive and preferably at least one active ingredient and/or further auxiliary agents.

For further processing of a laminate, it is usually necessary to remove one of the films, in particular the second film, from the laminate. For example, it happens that the laminate is rolled up into a laminate roll in a manufacturing step, wherein one of the films, preferably the second film, serves as an intermediate and/or protective film. For example, it may be necessary for rolling up to protect the formulation arranged on the carrier film by such a cover film. For further processing, in particular by means of another machine and/or at another place and/or at another time, it may then be necessary to remove said cover film. The cover film is removed in particular after the laminate roll has been unrolled. The film is preferably removed by peeling it off.

When removing the cover film, numerous problems arise. For example, when the cover film is removed, residues of the formulation may remain on the cover film and/or the removal may result in an uneven distribution of the formulation. Also, uneven lifting of the formulation may occur, in particular of the adhesive matrix containing the active ingredient. This may have a negative effect on the dosage of the active ingredient. In particular, it may happen that further processing of the matrix is impossible. These problems are particularly present when there is a change in the adhering forces between the formulation and the cover film, for example due to temperature influences, special properties of the formulation or a change in the adhering characteristics of the cover film, for example a degradation of silicone in the case of a siliconized cover film. Furthermore, problems arise when film materials with similar adhering characteristics are chosen.

### SUMMARY OF THE INVENTION

It is an object of the invention to provide a device for manufacturing an adherent film composite, preferably a TTS film composite, wherein the removal a film from a laminate is improved.

According to the invention, the object is achieved with a method for manufacturing an adherent film composite as described herein and a device for manufacturing an adherent film composite as described herein.

The method for manufacturing an adherent film composite according to the invention is preferably a method for manufacturing a TTS film composite. The film composite to be manufactured comprises in particular at least one film and a formulation, preferably comprising an active ingredient and/or adhesive. Adherent means in particular adhesive. When manufacturing a TTS film composite, it is preferred that the film composite comprises at least one carrier film and a matrix arranged thereon. The first step of the method consists in providing a laminate. The laminate is in particular a TTS laminate. It is preferred that the laminate to be provided is a laminate sheet, preferably a laminate film sheet. A preferred alternative designation for laminate sheet is laminate band. In particular, the laminate is provided by a laminate roll. The laminate, in particular the laminate sheet, is preferably provided in a wound from. The laminate sheet preferably has a length of at least 1 m, preferably at least 5 m, particularly preferably at least 10 m. Preferably, the laminate sheet has a length to width ratio of at least 2:1, preferably at least 5:1, particularly preferred at least 10:1. The laminate comprises a first film, a second film, and an adherent formulation arranged between the first film and the second film. In particular, the first film is a carrier film, also called intermediate liner or release liner. It is preferred that the first film comprises PET, in particular consists thereof. The second film is preferably a cover film, also called backing layer. The second film preferably comprises paper and/or polyethylene, in particular consists thereof. It is particularly preferred that the second film is a paper film coated with polyethylene. In particular, the first and/or the second film are siliconized. It is preferred that the formulation comprises an active ingredient and/or adhesive. A second step of the method consists of feeding the laminate in a feeding direction. Feeding here means in particular guiding the laminate to a separation area where the films of the laminate are separated. A further step consists of separating the first film with the formulation arranged thereon from the second film. The separation preferably takes place in a separation area. In particular, it is a peel-off separation, so that the first film is peeled off the second film. Here, it is preferred that the separation takes place in such a way that after the separation the formulation is arranged on the first film, wherein it is particularly preferred that after the separation the second film is substantially free of formulation. The separation of the first film from the second film is achieved by feeding out the first film from the second film obliquely to the feeding direction. The second film is passed on in the feeding direction. Furthermore, a rotation of the first film about its own longitudinal axis (of the first film) takes place when the first film is fed out. The first film is fed out in a feeding-out direction. When feeding out the first film obliquely to the feeding direction, it is preferred that the first film bends away from the second film and/or the feeding direction. In other words, the first and second films are separated by folding the first film in the separation area, in particular by 90°. This folding can be described in other words, in particular, based on folding a DIN A4 paper, for example, wherein a corner of the DIN A4 paper is folded inwards beyond the edge of the paper.

Preferably, the folding edge has a 45° angle. Feeding out then takes place in the direction of that short side of the DIN A4 sheet which has the aforementioned corner of the DIN A4 sheet. It is preferred that the fed out first film with formulation arranged thereon is the film composite to be manufactured. Preferably, the method is carried out as a continuous method.

Surprisingly, due to the oblique, in particular at a 90° angle, feeding out the first film with formulation arranged thereon, it is advantageous that during separation the formulation adheres substantially completely to the first film and thus substantially no formulation remains on the second film.

In a preferred embodiment, the first film is obliquely fed out at an angle, also called the feeding-out angle, of more than 70°, preferably of 70°-110°, particularly preferably of approx. 90°, relative to the feeding direction. If the first film is fed out at a particularly preferred angle of 90°, it is thus preferred that the feeding-out direction of the first film is perpendicular to the feeding direction of the laminate, or the first fed-out film is perpendicular to the laminate.

It is preferred that the rotation of the first film about its own longitudinal axis is at least 60°, preferably 90°-270°, and particularly preferred approx. 180º. The 180º rotation can be described in other words in such a way that before the rotation the films are located at the front, while after the rotation they are located at the rear, wherein it is particularly preferred that before the rotation a front side of the first film is at the top, while after the rotation the rear side of the first film is at the top.

In a preferred embodiment, the laminate is fed in a feeding plane, the first film is fed out in a feeding-out plane and the second film is preferably passed on after separation in a passing plane. It is preferred that at least two of the feeding plane, the passing plane and the feeding-out plane are parallel, in particular identical to each other. In other words, at least two of the following elements are guided in parallel, in particular identically: laminate, fed-out first film and passed on second film. It is particularly preferred that the laminate and the passed on second film are guided in the same plane and in the same direction.

It is preferred that the formulation comprises an active ingredient and/or an adhesive. The active ingredient in particular comprises a transdermal active ingredient. The adhesive in particular comprises a skin adhesive.

In a preferred embodiment, at least one of the film is adherence-reduced. This adherence reduction is implemented in particular in such a way that at least one of the films is siliconized, i.e. coated with silicone.

On the one hand, it is possible that the first and second films have substantially equal adherence and/or that the adhesion of the matrix to the two films is substantially equal. This equal adherence means in particular an equal adherence or adhesion between the second film and the formulation compared to the adherence or adhesion between the first film and the formulation. On the other hand, it is preferably possible that the second film has a lower adherence than the first film. In other words, the second film has a greater adherence reduction than the first film. This lower adherence means in particular a lower adherence or adhesion between the second film and the formulation or a greater lower adherence or adhesion between the first film and the formulation. Due to the different adherence of the films, it is in particular advantageously implemented that the formulation adheres to the first film, in particular when separating the films, while it detaches from the second film.

It is preferred that the laminate is fed by pulling, in particular rolling up the second film. Here, it is particularly preferred that the entire laminate is pulled first, wherein after separating the first film with formulation arranged thereon from the laminate, only the second film is continued to be pulled. This pulling is preferably done by means of a tension reel. It is preferred that the first film is fed out by pulling, in particular by rolling up the first film. Pulling by means of a tension reel is particularly preferred. While tension reels are used for pulling the laminate or the second film as well as for pulling the first film, it is preferred that the pulling directions of the tension reel are perpendicular to each other and/or in an identical plane.

In a preferred embodiment, the first film is fed out along a bending edge which is preferably angular.

It is preferred that the rotation of the first film about its own longitudinal axis is achieved by bending the film at the bending edge.

In particular, the method is carried out with a device having one or more features of the device according to the invention described below.

The device according to the invention for manufacturing an adherent film composite is preferably a device for manufacturing an adherent TTS film composite. The device has a laminate conveying means for guiding a laminate in a feeding direction. In particular, the laminate is configured as a laminate described above in the context of the method. Preferably, the laminate conveying means is configured to feed the laminate to a separation area for separating the laminate. Further, the device comprises a film composite conveying means for guiding a first film of the laminate in a feeding-out direction. The film composite conveying means is configured in particular to feed out the first film of the laminate starting from the separation area. It is preferred, that the feeding direction and the feeding-out direction are not the same and are in particular perpendicular to each other. The device further comprises a separating device for separating the first film from the laminate. In particular, the separating device is configured for peel-off separation of the first film from the laminate. It is preferred that the separating device is configured to separate the first film with formulation arranged thereon from the second film of the laminate. According to the invention, the separating device is configured such that, for separation, the first film is bendable about the separating device so that the first film is fed out obliquely to the feeding direction and so that the first film is twisted in the longitudinal direction. Preferably, the fed out first film with formulation arranged thereon is the film composite to be manufactured.

In a preferred embodiment, the separating device has a bending edge. It is preferred that it is a pointed and/or angular bending edge. On the other hand, it is possible that the bending edge is round. To form the bending edge, which is in particular angular, it is preferred that the separating device has a bending plate. To form a round bending edge, it is possible that the separating device has a roller, which is preferably rotatable, or a full or partial cylinder as a bending edge. It is preferred that the separating device has a squeegee, preferably consists thereof, in particular with a bending edge.

In particular, the bending edge is arranged opposite and/or adjacent to a feeding path of the laminate on which the laminate is fed by the laminate conveying means. It is particularly preferred that the bending edge is arranged such that it comes into contact with the laminate being fed. This contact of the laminate with the bending edge preferably takes place on a side of the bending edge that is opposite a side of the fed-out first film.

In a preferred embodiment, the bending edge is arranged at an angle of 30° to 60°, particularly preferably at an angle of 45°, to the feeding direction.

It is preferred that the bending edge is arranged parallel to a feeding plane of the laminate and/or parallel to a feeding-out plane of the first film and/or parallel to a passing plane of the second film of the laminate. Particularly preferably, this parallel arrangement of the bending edge is parallel to one or more of the aforementioned planes, so that there is a distance to the plane(s).

It is preferred that the device is configured such that the feeding-out direction is oblique to the feeding direction. Here, it is particularly preferred that oblique means an angle of 60° to 120° and particularly an angle of 90°. In order to implement this oblique arrangement of the feeding-out direction to the feeding direction, it is preferred that the conveying direction of a reel of the laminate conveying means is correspondingly arranged obliquely to the conveying direction of a reel of the film composite conveying means. Particularly preferably, the conveying directions of this reel are perpendicular and/or in a common plane to each other.

It is preferred that the laminate conveying means has a reel, in particular a tension reel. Preferably, the tension reel of the laminate conveying means rolls up a second film of the laminate to be fed and/or the laminate to be fed. Alternatively or in addition to the reel of the laminate conveying means, it is preferred that the film composite conveying means has a reel, in particular a tension reel. In particular, the tension reel of the film composite conveying means rolls up a first film of the laminate. In a preferred embodiment, the device for manufacturing an adherent film composite further comprises a reel, in particular a payoff reel, for unrolling the laminate. It is particularly preferred that the tension reel of the laminate conveying means pulls the second film of the laminate, and the tension reel of the film composite conveying means pulls the first film with formulation of the laminate arranged thereon and thus unrolls the laminate from the payoff reel. In this embodiment with two tension reels and one payoff reel, it is preferred that the conveying direction of the tension reel of the laminate conveying means is identical to the conveying direction of the payoff reel, wherein the conveying direction of the tension reel of the film composite conveying means is oblique, in particular perpendicular to this direction, wherein it is particularly preferred that the three conveying directions are arranged in a single plane.

In a preferred embodiment, the device for manufacturing an adherent film composite has at least one of the features of the method according to the invention described above, wherein it is particularly preferred that the device is configured to perform a method according to the invention described above.

### BRIEF DESCRIPTION OF THE DRAWINGS

In the following, the invention is described in more detail by means of preferred embodiments with reference to the accompanying drawings.

In the drawings:

FIG. 1a is a schematic side view of a laminate,

FIG. 1b is a schematic exploded view of a laminate,

FIG. 2a is a schematic side view of a film composite,

FIG. 2b is a schematic exploded view of a film composite,

FIG. 3 is a schematic plan view of an embodiment of a device for manufacturing an adherent film composite according to the invention,

FIG. 4 is a schematic, perspective view of a further embodiment of a device for manufacturing an adherent film composite according to the invention.

### DESCRIPTION OF THE INVENTION

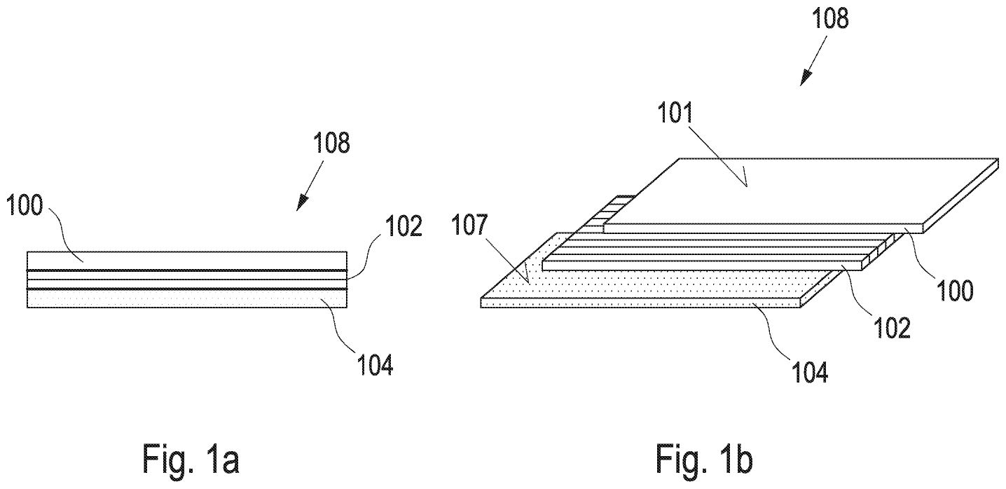

FIG. 1a shows an example of a type of standard laminate 108 which can be used as a starting basis for the method according to the invention for manufacturing an adherent film composite 106.

The illustrated laminate 108 comprises a first film 100, a second film 104 and a formulation 102 arranged between the first film 100 and the second film 104. Preferably, laminate 108 is a TTS laminate. In particular, the first film 100 is a carrier film. The second film 104 is preferably a cover film. Formulation 102 is preferably a matrix, which particularly preferably comprises an adhesive as well as a transdermal active ingredient.

Alternative embodiments of a laminate 108 may have further layers, such as further films or formulations.

FIG. 1b shows an exploded illustration of laminate 108 of FIG. 1a.

The first film 100 has a surface 101 with a first adhering characteristic, while the second film 104 has a surface 107 with a second adhering characteristic. It is preferred that the first adhering characteristic of the first film 100 is different than the second adhering characteristic of the second film 104. It is particularly preferred that the first film 100 has a greater adherence than the second film 104. It is preferred that the first film 100 and/or the second film 104 is adherence-reduced, in particular the first film 100 has a lower adherence reduction that the second film 104. Adherence here means in particular an adhesive force between the first film 100 and formulation 102 or the second film 104 and formulation 102, respectively. Particularly preferably, the adhesive force between the first film 100 and formulation 102 is greater than the adhesive force between the second film 104 and formulation 102. Due to these different adherences of the first film 100 to the second film 104, it is preferably advantageous that when the films 100; 104 are separated, formulation 102 adheres to and thus remains on the first film 100.

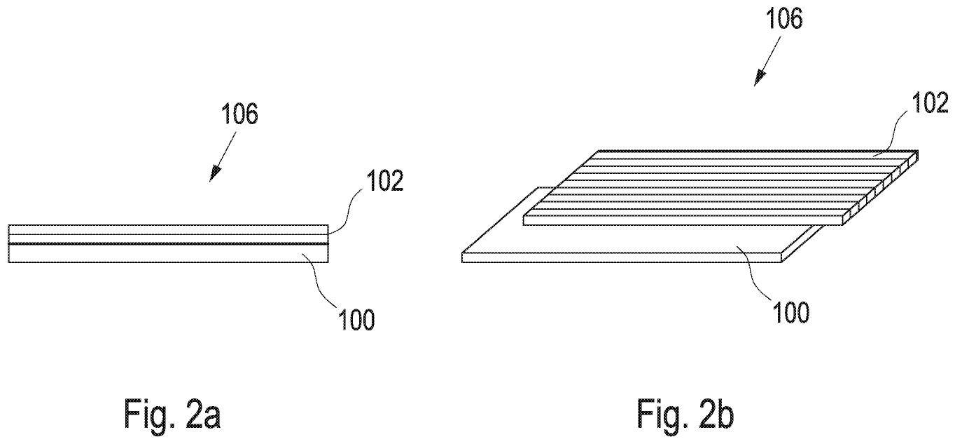

FIG. 2a exemplarily shows a film composite 106, which is preferably a film composite 106 to be manufactured by the method according to the invention and/or the device according to the invention.

Film composite 106 substantially corresponds to laminate 108 of FIG. 1a, with the second film 104 having been removed or separated therefrom, in particular peeled off. In addition, compared to the embodiment in FIG. 1a, a rotation about its own longitudinal axis, in this case by 180°, was made. Thus, the first film 100 is shown at the bottom, wherein formulation 102 is arranged above the first film 100, in particular adherently connected thereto.

In a subsequent use, for example, formulation 102 may be arranged on a skin for transdermal administration, preferably with the first film 100 covering the formulation to the other side.

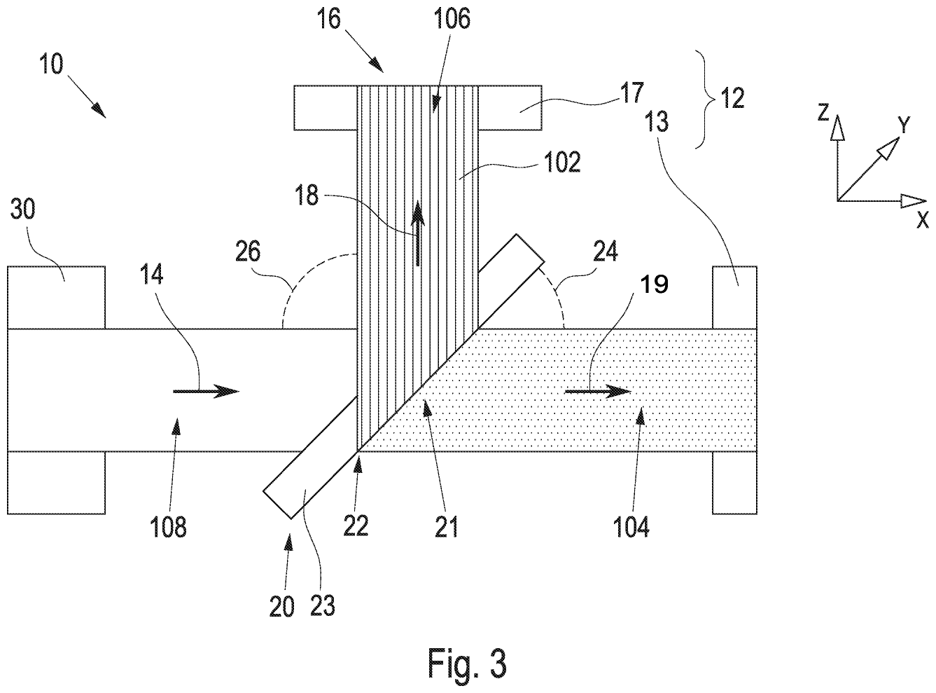

FIG. 3 shows an embodiment of device 10 for manufacturing an adherent film composite 106 according to the invention. Preferably, the illustrated film composite 106 is the film composite 106 illustrated in FIG. 2a.

A laminate 108 is provided on a payoff reel 30. Preferably, laminate 108 is the laminate 108 illustrated in FIG. 1a.

Laminate 108 is fed in the feeding direction (illustrated by arrow 14) by laminate conveying means 12. As illustrated, laminate conveying means 12 has two tension reels 13; 17. The first tension reel 17 winds the adherent film composite 106 to be manufactured, consisting of film 100 with formulation 102 arranged thereon. Accordingly, the first tension reel 17 is also part of film composite conveying means 16 for conveying the first film 100 and formulation 102 arranged thereon. The second tension reel 13 winds up the second film 104 of laminate 108. By pulling the films 100; 104 of laminate 108 by means of the tension reels 13; 17, laminate 108 is fed, so that laminate 108 is unwound from payoff reel 30.

In an alternative embodiment, which is not illustrated, it is possible, for example, to use only the first tension reel 17 as laminate conveying means 12 and as film composite conveying means 16. In particular, the second reel would thus be omitted in this alternative embodiment. Since the second film 104 is often an intermediate product or waste product, it could thus be ejected without rewinding. In this alternative embodiment, laminate conveying means 12 would thus correspond to film composite conveying means 16, wherein laminate conveying means 12 or film composite conveying means 16 comprises the first tension reel 17.

As shown in FIG. 3, the first tension reel 17 conveys the first film 100 in feeding-out direction 18. The second tension reel 13 conveys the second film 104 in passing direction 19. It is preferred that at least one and more preferably all of the following conditions apply: feeding direction 14 is parallel, preferably identical to passing direction 19; feeding-out direction 18 is obliquely, in particular at a 90° angle to feeding direction 14 and/or to passing direction 19; the feeding path of laminate 108 is congruent with the passing path of the second film 104; the first film 100 is fed out in a feeding-out plane (shown in the xy plane), this plane being parallel, in particular identical to the feeding plane of laminate 108 and/or to the passing plane of the second film 104.

In the illustrated embodiment, a separation, preferably a peel-off separation, of the first film 100 with formulation 102 arranged thereon from the second film 104 of laminate 108 takes place in a separation area 21. Here, the first film 100 is bent around a bending edge 22 of a separating device 20. In the illustrated embodiment, separating device 20 comprises in particular a bending plate 23 which forms bending edge 22. The bending edge has a bending angle 24 which is preferably 45° to feeding direction 14 and/or 45° to passing direction 19 and/or 45° to the fed laminate 108 and/or 45° to the passed on second film 104.

At bending edge 22, the first film 100 of laminate 108 is folded or folded over such that, after separation, the bottom of the first film 100 with formulation 102 arranged thereon faces upwards. During separation, the first film 100 is thus rotated 180º about the longitudinal axis of film 100. The first film 100 with formulation 102 arranged thereon is fed out after separation in feeding-out direction 18 at a feeding-out angle 26. The feeding-out angle 26 is preferably 90° to feeding direction 14 and/or passing direction 1 and/or the fed laminate 108 and/or the fed-out second film 104.

Separating edge 22 is preferably arranged parallel to the feeding plane of laminate 108 and/or to the passing plane of the second film 104 and/or to the feeding-out plane of the first film 100. It is particularly preferred that bending edge 22 is arranged such that the fed laminate 108 is guided in an opposite and adjacent manner, in particular guided in contact.

Surprisingly, the oblique feeding out of the first film 100 with formulation 102 arranged thereon, particularly at a 90° feeding-out angle 26, results in substantially complete adherence of formulation 102 to the first film 100 during separation, and thus substantially no formulation 102 remains on the second film 104.

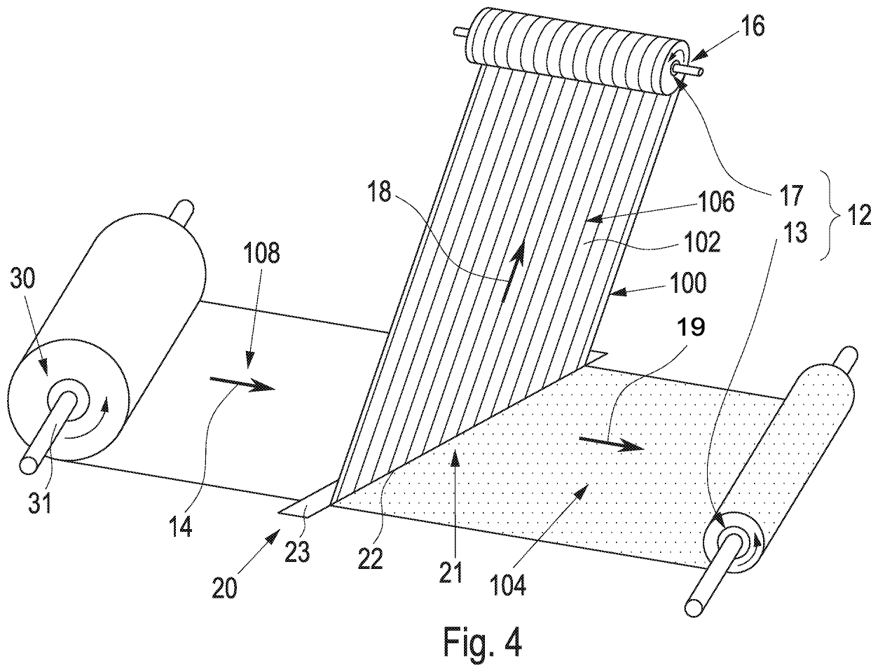

FIG. 4 shows a further embodiment of a device for manufacturing an adherent film composite 106 according to the invention, wherein the embodiment substantially corresponds to that of FIG. 3.

In contrast to the embodiment in FIG. 3, in FIG. 4 the reels 13; 17; 30 are arranged in a twisted manner so that the direction of rotation of the reels 13; 17; 30 (shown by the rotation arrows) also rotate in the opposite direction.

1. A method for manufacturing an adherent film composite, preferably a TTS film composite, comprising the following steps:
providing a laminate, the laminate comprising a first film, a second film, and an adherent formulation arranged between the first film and the second film,
feeding the laminate in a feeding direction, and
separating, preferably in a peeling manner, the first film with the formulation arranged thereon from the second film,
wherein the films are separated by the first film being fed out obliquely to the feeding direction from the second film,
wherein the second film is passed on in the feeding direction, and
wherein the first film rotates about its longitudinal axis when feeding out the first film.

2. The method according to claim 1, wherein the first film is fed out at an angle of more than 60°, preferably from 60° to 120°, particularly preferred approx. 90°, relative to the feeding direction.

3. The method according to claim 1, characterized in that the rotation of the first film about its own longitudinal axis is at least 60°, preferably 90°-270°, and particularly preferred approx. 180°.

4. The method according to claim 1, wherein the laminate is fed in a feeding plane, the first film is fed out in a feeding-out plane and, preferably, the second film is passed on after separation in a passing plane, wherein at least two of the feeding plane, the passing plane and the feeding-out plane are parallel, in particular identical to one another.

5. The method according to claim 1, wherein the formulation comprises an active ingredient and/or an adhesive.

6. The method according to claim 1, wherein at least one of the films is adherence-reduced, preferably coated with silicone.

7. The method according to claim 1, wherein the second film has a lower adherence than the first film; or the first film has a lower adherence than the second film; or the first film and the second film substantially have the same adherence.

8. The method according to claim 1, wherein the method is performed with a device.

9. A device for manufacturing an adherent film composite, preferably a TTS film composite, comprising
a laminate conveying means for guiding a laminate in a feeding direction,
a film composite conveying means for guiding a first film of the laminate in a feeding-out direction, and
a separating device for separating, preferably in a peeling manner, the first film from laminate,
wherein the separating device is configured such that the first film; is bendable about the separating device so that the first film is fed out obliquely to the feeding direction, and so that the first film is twisted in the longitudinal direction.

10. The device for manufacturing an adherent film composite according to claim 9, wherein the separating device has a bending edge, which is in particular angular, wherein the bending edge is preferably arranged adjacent to a feeding path of the laminate by the laminate conveying means.

11. The device for manufacturing an adherent film composite according to claim 10, wherein the bending edge is arranged at an angle of 30° to 60°, particularly preferred at an angle of approx. 45°, to the feeding direction.

12. The device for manufacturing an adherent film composite according to claim 10, wherein the bending edge is guided parallel to a feeding plane of the laminate and/or to a feeding-out plane of the first film and/or to a passing plane in which a second film of the laminate is guided.

13. The device for manufacturing an adherent film composite according to claim 9, wherein the device is configured such that the feeding-out direction is oblique, preferably at an angle of 60° to 120°, particularly preferred at an angle of approx. 90° to the feeding direction.

14. (canceled)
